(19) United States
(12) Patent Application Publication
(10) Pub. No.: US 2005/0084522 A1
(43) Pub. Date: Apr. 21, 2005

(54) Cationic lipids for nuclei acid delivery

(76) Inventor: Michalakis Savva, Roselle Park, NJ (US)

Correspondence Address: MICHALAKIS SAVVA, 240 WEST SUMNER AVENUE, APT. 38, ROSELLE PARK, NJ, 07204, US

(21) Appl. No.: 10/686,262

(22) Filed: Oct. 15, 2003

(51) Int. Cl. A61K 48/00; A61K 9/127; C12N 15/74

(52) U.S. Cl. 424/450; 435/488; 514/44; 554/37

(57) ABSTRACT

The invention describes the synthetic methods for a series of pH-sensitive cationic lipids with diamido linkages between the 1,2-diamino-3-propanol backbone and the hydrocarbon chains. Their in vitro biological activity of the resulting lipid-DNA complexes is also described.

### BACKGROUND OF THE INVENTION

Lipid-based drug delivery systems that can be constructed to respond to acidic environments are of great interest to medical sciences, as they can be potentially targeted to release their contents to hyperactive tumor tissues of low pH or to respond by fusion and disruption of primary endosomes, thereby releasing their contents into the cytoplasm. Cationic lipids, is a particularly attractive alternative to the viral vectors, because they do not induce any humoral responses. Moreover, it is possible to design and synthesize a wide variety of cationic lipids that depending on their structural features, promote gene expression by affecting one or more of the essential requirements for transfection, i.e. efficient compaction of plasmid DNA, lipoplex internalization, fusion of the internalized lipoplex with lysosome membrane and translocation of the plasmid DNA to the nucleus.

The present invention describes the synthesis and use of novel cationic lipids for nucleic acid and other macromolecular drug delivery in vitro and in vivo.

Herein, we report on the synthesis and transfection activity of novel cationic lipids of the 1,2-diamino-3-propanol series, in which the polar headgroup bis-(2-dimethylaminoethane) is attached via a carbamate spacer at the 3-position of the 1,2-diaminopropan-3-ol, whereas the hydrophobic alkyl chains are bonded to the Nitrogen atoms at the 1- and 2-position of the diaminopropanol backbone through acyl linkers.

### SUMMARY OF THE INVENTION

The inventor has proposed that cationic lipids of general structure S when dispersed in aqueous solvent can be used as carriers for transfer of nucleic acids, other macromolecules and synthetic drugs into cells.

The structure S of cationic lipids comprises a monovalent or bivalent polar head that could be a primary, secondary, tertiary or a quaternary amine or a guanidine group. Cationic lipids of structure S are double-chained derivatives bearing saturated or unsaturated chains that are linked to the propylene synthon at 1 and 2 position. The polar head is linked to the hydrophobic part of the molecule through a connector as shown in structure S.

Assemblies of structure S can associate or encapsulate therapeutic compounds and carry them inside the cells.

### DESCRIPTION OF FICURES

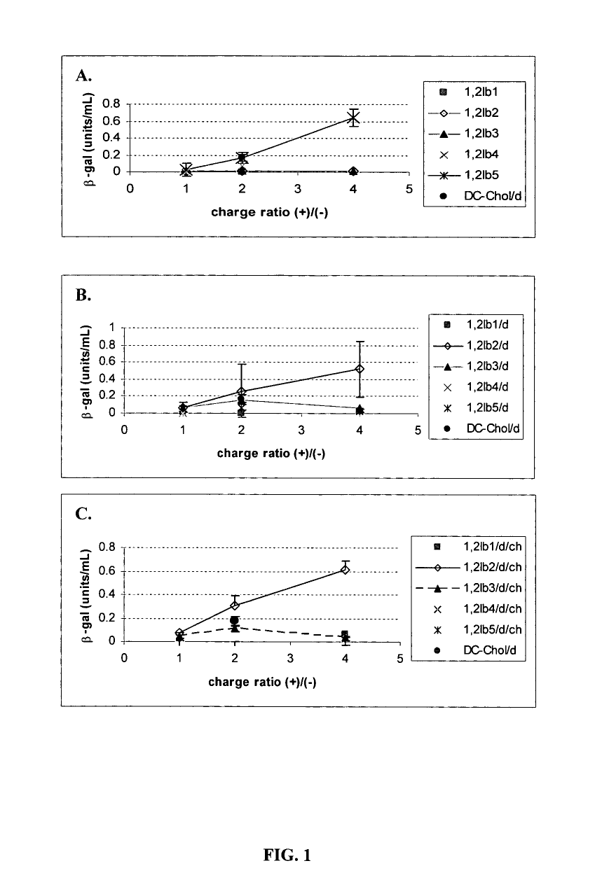

FIG. 1 Transfection activity of cationic lipids in B16F0 cells. A. 1,2lb lipoplexes B. 1,2lb/d lipoplexed C. 1,2lbd/ch lipoplexes. The o-nitrophenol formation was converted to units, using a standard curve obtained with commercial β-galactosidase. The data presented are the average of values obtained from seven wells (n=7), of two independent experiments performed at different times.

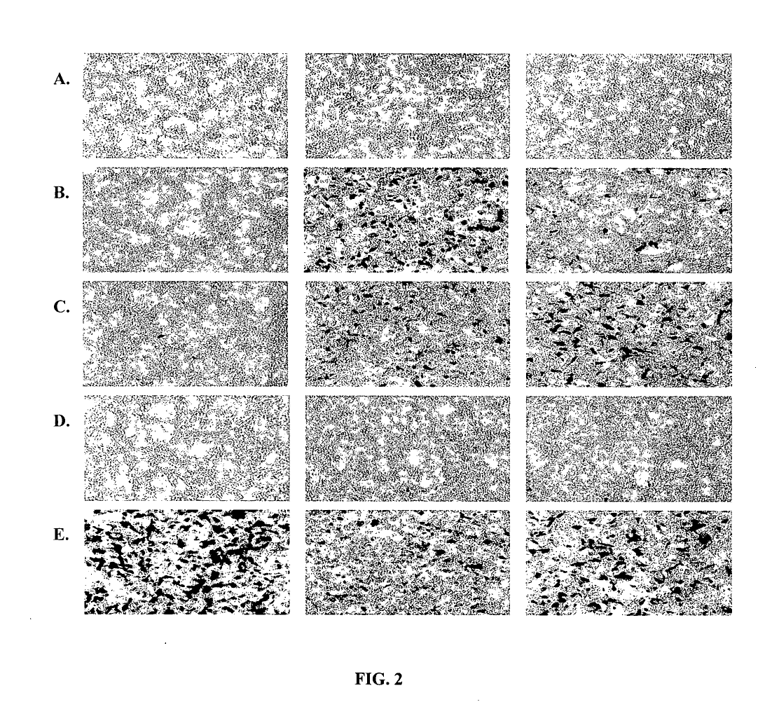

FIG. 2 Transfection activity of cationic lipids against B16F0 murine melanoma cells, in the absence of serum. From left-to-right, lipid, lipid/dope (3:2 mol/mol), lipid/dope/cholesterol (3:2:1 mol/mol). The (+)/(−) charge ratio was fixed to 2. A. R═C₁₁H₂₃, B. R═C₁₃H₂₇, C. R═C₁₅H₃₁ D. R═C₁₇H₃₅, E. R═C₁₇H₃₁. R₁═CH₂CH₂N(CH₃)₂, R₂═CH₃, R₃═CH₃, R₄═H.

### DESCRIPTION OF THE INVENTION

The present invention describes the synthesis of novel cationic lipids and their application as nucleic acid and other drug transfer vehicles in vitro and in vivo. Cationic lipids of general structure S described in claim 1 were synthesis and their potential to carry nucleic acid molecules in cells evaluated and presented in FIGS. 1 and 2. Compounds of the structure S are efficient transfection reagents due to their capability to produce complexes with nucleic acid molecules.

The synthesis of the invented compounds is carried out according to page 1 and page 2 of Scheme I. Quaternization of the final compounds was effected by refluxing with methyl iodide.

### EXAMPLE 1

### Synthesis of Bis-(2-dimethylamino-ethyl)-amine

Solid bis-(2-chloroethyl)-amine hydrochloride (17.85 g; 0.10 mol) was dissolved in a 500 ml round bottom flask with addition of 152 ml dimethylamine (54 g; 1.20 mole). After a 72 hr rigorous stirring at room temperature the reaction was made alkaline with 6M NaOH (100 ml) and further saturated with anhydrous potassium carbonate. Upon alkalization an oily liquid separates on the upper layer. The mixture was stirred for one half hour and then transferred to a 500 ml separation funnel where the crude product was extracted with 4×150 ml of ethyl ether (ninhydrin test of the organic phase turns negative). The extracts were collected and dried (MgSO₄) for overnight. MgSO₄ was removed by suction filtration and the organic solvent was removed under diminished pressure at 45° C. to give a total of 5.0 g of a colorless oil yield (31%). Further purification of the triamine was performed by chromatography with a 50% recovery (Kupchan et al., 1971). MS (FAB & ES) m/z 160.2 [M+H]⁺; ¹H NMR (400 MHz, CDCl₃, 20°C., TMS) δ 2.60-2.63 (t, 4H, J=6.2 Hz, NCH₂CH₂N(CH₃)₂), 2.31-2.34 (t, 4H, J=6.2 Hz, CH₂N(CH₃)₂), 2.13 (s, 12H, N(CH₃)₂); ¹³C NMR (100 MHz, CDCl₃, 20° C., TMS) δ 59.21 (2CH₂), 47.48 (2CH₂), 45.60 (4CH₃).

### EXAMPLE 2

### Synthesis of methyl 2,3-diaminopropionate dihydrochloride (2)

2,3-diaminopropionic acid monohydrochloride (2.0 g; 14.2 mmole) was pulverized and transferred to a 500 ml round bottom flask containing 50 ml anhydrous methanol. Bound water was removed by evaporating the solvent several times at low pressure at 75° C. The dry powder was finally dissolved in fresh anhydrous methanol (100 ml) saturated with anhydrous hydrogen chloride gas. The reaction was refluxed at 85-90° C. for 17 h. Methanol was eliminated under diminished pressure to give 2.85 g as a yellowish crystal, 64-83% purity, as determined by ¹H NMR. R_{f}=0.86 (A). ¹H NMR (400 MHz, D₂O, 20° C., TMS) δ 4.26-4.34 (t, 1H, CH₂CH(NH₂)CO), 3.66 (s, 3H, COOCH₃), 3.22-3.44( m, 2H, CH₂CH).

### EXAMPLE 3

### Synthesis of Methyl 2,3-dilauroylamidopropionate (3)

Methyl 2,3-dilauroylamidopropionate was synthesized with a procedure similar to that described by Sunamoto and coworkers (Sunamoto et al., 1990). Briefly, to a 100 ml anhydrous DMF solution of methyl 2,3-diaminopropionate dihydrochloride (1.36 g; 7.11 mmole) in a 250 ml round bottom flask was added 10.0 ml TEA (71.1 mmole) immediately followed by addition of 7.0 ml myristoyl chloride (28.4 mmole). The reaction was stirred at 60° C. for 6 h. DMF was evaporated with the aid of a rotary evaporator at 90° C. under reduced pressure and the crude material was transferred with 100 ml CHCl₃ to a 500 ml separation funnel where it was washed once with HCl and once with Na₂CO₃, respectively. The organic phase was collected in a 250 ml round bottom flask and the CHCl₃ was removed under reduced pressure with the aid of a rotary evaporator. The dried crude was dissolved in a minimum amount of hot methanol and left overnight for crystallization. The product was collected by suction filtration as a white crystalline powder (2.1 g, 61.3%). R_{f}=0.76 (C). ¹H NMR (400 MHz, CDCl₃, 20° C., TMS) δ 6.89 (d, 1H, HNCH), 6.25 (t, 1H, HNCH₂), 4.57 (m, 1H, CHCOOCH₃), 3.72 (s, 3H, COOCH₃), 3.60 (t, 2H, HNCH₂CH), 2.11-2.21 (m, 4H, COCH₂), 1.58 (m, 4H, COCH₂CH₂), 1.21 (b, 32H, (CH₂)₈CH₃), 0.82-0.85 (t, 6H, CH₃).

### EXAMPLE 4

### Synthesis of 2,3-dilauroylamidopropan-3-ol (8)

To a 100 ml solution of methyl 2,3-dilauroylamidopropionate ( 3.52 g; 7.29 mmole) in absolute ethanol in a 250 ml round bottom flask was added 10.9 ml (21.87 mmole) of 2.0 M lithium borohydride solution in THF. Hydrogenation was performed in a dry N₂ gas environment at 50-60° C. for 3 h and then at 23° C. for an additional 12 h. Ethanol was removed and the crude material was taken with 100 ml chloroform, transferred to a 500 ml separation funnel and washed once with 100 ml 1.0 N HCl and with Na₂CO₃, respectively. The organic phase was concentrated to an oil of high viscosities, which was applied to a silica gel column (3.2×30 cm). Elution was performed with 100 ml CHCl₃, 1, 1.5, 2, 2.5, 3, 10, 20 and 50% MeOH/CHCl₃. Fractions between 2 and 20% were pooled and concentrated to give a total of 2.94 g (89%) 2,3-dilauroylamidopropanol as a white powder. R_{f}=0.27 (D). ¹H NMR (400 MHz, CDC1₃, 20° C., TMS), δ 6.25-6.50 (b, 2H, HNCH₂HNCH), 3.60-3.90 (m, 2H, CH(NH)CH₂OH), 3.40-3.55 (m, 2H, HNCH₂CH), 3.20-3.30 (m, 1H, CH), 2.12-2.22 (m, 4H, COCH₂), 1.59 (b, 4H, COCH₂CH₂), 1.23 (b, 32H, (CH₂)₈CH₃), 0.83-0.86 (t, 6H, CH₃).

### EXAMPLE 5

### Synthesis of 2,3-dilauroylamidopropane-1-(p-nitrophenyl) carbonate (13)

2,3-dilauroylamidopropanol (2.94 g; 6.47 mmole) was suspended with continuous stirring in 100 ml anh. THF, in a 250 ml round bottom flask, kept at 25° C. with the aid of an oil bath. To the suspension was added 4-nitrophenyl chloroformate (1.30 g; 6.47 mmole) followed by a dropwise addition of pyridine (0.52 ml; 6.47 mmole). The reaction was stopped after 5 h. THF was removed under diminished pressure and the crude was transferred with 100 ml chloroform to a 500 ml separation funnel and washed once with 100 ml and 1.0 N HCl and Na₂CO₃, respectively. The organic phase was concentrated to a viscous oil with the aid of a rotary evaporator and purified by chromatography on a silica gel column, eluting with 100 ml 1%, 2%, 3%, 4%, 5%, 20%, 50% MeOH/CHCl₃. Fractions between 3% and 20% were pooled and dried to give total of 3.9 g (6.28 mmole; 97% yield) 2,3-dilauroylamidopropane-1-(p-nitrophenyl) carbonate as a white crystalline material. R_{f}=0.49 (D). ¹HNMR (400 MHz, CDCl₃, 20° C., TMS) δ 8.27-8.24, 7.37-7.23 (two d, each J=9.1 Hz, 4H, C₆H₄), 6.64 (d, 1H, HNCH), 6.02 (t, 1H, HNCH₂), 4.18-4.40 (m, 3H, NHCHCH₂OCOO), 3.42-3.57 (m, 2H, HNCH₂CH), 2.14-2.20 (m, 4H, COCH₂), 1.57-1.79 (m, 4H, COCH₂CH₂), 1.0-1.24 (bs, 32H, (CH₂)₈CH₃), 0.82-0.90 (t, 6H, CH₃).

### EXAMPLE 6

### Synthesis of 1,2-dilauroylamidopropane-3-[bis-(2-dimethylaminoethane)]-carbamate (18; 1,2lb1)

To a solution of 2,3-dilauroylamidopropanane-1-(p-nitrophenyl) carbonate (3.9 g; 6.28 mmole) in 100 ml chloroform was added bis-(2-dimethylaminoethyl)-amine (1.7 ml; 9.42 mmole). The reaction was stirred at room temperature for 17 h. The reaction mixture was then transferred to a 500 ml separation funnel where it was washed once with 100 ml Na₂CO₃ 1.0 N. The organic phase was concentrated to an oil which was chromatographed on a silica gel column, eluting with 100 ml 2%, 4%, 200 ml 5%, 100 ml 7%, 10% and 200 ml 15%, 20%, 25%, 30%, 35%, 40% MeOH/CHCl₃. Fractions between 15% and 40% were pooled and dried up to obtain a total 2.95 g (73%). R_{f}=0.46 (E). Anal. Calcd for C₃₆H₇₃N₅O₄ (MW 639): C, 69.61; H, 11.42; N, 10.95. Found: C, 66.97; H, 11.77; N, 10.90. ¹H NMR (400 MHz, CDCl₃, 20° C., TMS), δ 7.15 (d, 1H, HNCH), 6.70 (t, 1H, HNCH₂), 4.13 (m, 3H, NHCHCH₂OCO), 3.20-3.50 (m, 6H, NHCH₂CH(NH)CH₂OCON(CH₂)₂—), 2.35-2.42 (m, 4H, CH₂N(CH₃)₂), 2.19-2.20 (d, 12H, N(CH₃)₂), 2.05-2.18 (m, 4H, COCH₂), 1.45-1.60 (m, 4H, COCH₂CH₂), 1.187 (b, 32H, (CH₂)₈CH₃), 0.80-0.90 (t, 6H, CH₃). ¹³C NMR (100 MHz, CDCl₃, 20° C.), δ 176.04 (NHCO), 175.34 (NHCO), 157.47 (OCO), 65.30, 58.63, 46.83, 37.75, 32.96, 30.68, 30.41, 27.35, 23.73, 15.17.

### EXAMPLE 7

### Synthesis of 1,2-dimyristoylamidopropane-3-[bis-(2-dimethylaminoethane)]-carbamate (19; 1,2lb2)

1,2lb2 was prepared analogously to 18 on a 1.5 mmole scale to give upon purification the product as a white solid (0.6 g, 0.87 mmole, 57%). R_{f}=0.76 (E). Anal. Calcd for C₄₀H₈₁N₅O₄ (MW 695): C, 69.06; H, 11.65; N, 10.07. Found: C, 68.74; H, 11.99; N, 9.98. MS (FAB) m/z 697.1 [M+H]⁺. ¹H NMR (400 MHz, CDCl₃, 20° C., TMS), δ 7.15 (d, 1H, HNCH) 6.70 (t, 1H, HNCH₂), 4.13 (m, 3H, CH(NH)CH₂OCO), 3.24-3.46 (m, 6H, NHCH₂CH(NH)CH₂OCON(CH₂)₂), 2.36-2.47 (m, 4H, CH₂N(CH₃)₂), 2.21-2.23 (d, 12H, N(CH₃)₂), 2.12 (m, 4H, COCH₂), 1.56-1.58 (m, 4H, COCH₂CH₂), 1.24 (b, 40H, (CH₂)₁₀CH₃), 0.80-0.90 (t, 6H, CH₃). ¹³C NMR (100 MHz, CDCl₃, 20° C.), δ 176.16 (NHCO), 175.34 (NHCO), 157.35 (OCO), 65.76, 58.63, 46.83, 46.37, 46.25, 37.69, 32.96, 30.71, 30.41, 27.05, 23.73, 15.20.

### EXAMPLE 8

### Synthesis of 1,2-dipalmitoylamidopropane-3-[bis-(2-dimethylaminoethane)]carbamate (20; 1,2lb3)

1,2lb3 was prepared similar to 1,2lb2 (white sticky powder, 1.9 g, 2.5 mmole, 58%). R_{f}=0.52 (E). Anal. Calcd for C₄₄H₈₉N₅O₄ (MW 751): C, 70.31; H, 11.85; N, 9.32. Found: C, 70.1; H, 11.97; N, 9.32. ¹H NMR (400 MHz, CDCl₃, 20° C., TMS), δ 7.15 (d, 1H, OCHNCH) 6.70 (t, 1H, OCHNCH₂CH), 4.13 (m, 3H, NHCHCH₂OCO), 3.20-3.50 (m, 6H, CH₂CHCH₂OCON(CH₂)₂), 2.40-2.50 (m, 4H, CH₂N(CH₃)₂), 2.22-2.25 (d, 12H, N(CH₃)₂), 2.10-2.20 (m, 4H, COCH₂), 1.45-1.60 (m, 4H, COCH₂CH₂), 1.21 (b, 48H, (CH₂)₁₂CH₃), 0.82-0.86 (t, 6H, (CH₂)₁₂CH₃). ¹³C NMR (100 MHz, CDCL₃, 20° C.), δ 176.04 (NHCO), 175.34 (NHCO), 157.44 (OCO), 65.67, 58.61, 46.83, 46.58, 37.72, 32.96, 30.74, 30.41, 26.74, 23.73, 15.20.

### EXAMPLE 9

### Synthesis of 1,2-distearoylamidopropane-3-[bis-(2-dimethylaminoethane)]carbamate (21; 1,2lb4)

1,2lb4 was prepared similarly to 1,2 lb3 with bis-(2-dimethylaminoethyl) amine and gave the product as a half-white sticky solid (0.6 g, 0.7 mmoles, 48%). R_{f}=0.48 (E). Anal. Calcd for C₄₈H₉₇N₅O₄ (MW 807): C, 71.37; H, 12.02; N, 8.67. Found: C, 70.37; H, 11.92; N, 8.18. ¹H NMR (400 MHz, CDCl₃, 20° C., TMS), δ 7.00 (d, 1H, HNCH), 6.67 (t, 1H, HNCH₂CH), 4.13 (m, 3H, NHCHCH₂OCON), 3.33 (m, 6H, CH₂CHCH₂OCON(CH₂)₂—), 2.10-2.40 (m, 20H, CH₂N(CH₃)₂; COCH₂CH₂(CH₂)₁₄CH₃), 1.55-1.57 (m, 4H, COCH₂CH₂), 1.22 (s, 56H, (CH₂)₁₄CH₃), 0.84 (t, 6 H, CH₃).

### EXAMPLE 10

### Synthesis of 2,3-dioleoylamidopropane-1-[bis-(2-dimethylaminoethane)] carbamate (22; 1,2lb5)

1,2lb5 was prepared similarly to 1,2lb4 with bis-(2-dimethylaminoethyl) amine and gave the product as a yellow oil (0.9 g, 1.1 mmoles, 35%). R_{f}=0.5 (E). Anal. Calcd for C₄₈H₉₃N₅O₄ (MW 807): C, 71.73; H, 11.58; N, 8.71. Found: C, 71.03; H, 11.85; N, 7.73. ¹H NMR (400 MHz, CDCl₃, 20° C., TMS), δ 7.3 (b, 1H, HNCH), 6.89 (b, 1H, HNCH₂), 5.28 (m, 4H, CH═CH), 4.16 (m, 3H, HNCHCH₂OCO), 3.39 (m, 6H, HNCH₂(HN)CHCH₂OCON(CH₂—)₂), 1.97-2.42 (m, 28H, COCH₂; CH₂CH═CHCH₂; CH₂N(CH₃)₂), 1.21-1.55 (m, 44H, COCH₂CH₂(CH₂)₄CH₂CH═CHCH₂(CH₂)₆),0.80-0.83 (t, 6H, CH₂CH₃). ¹³C NMR (100 MHz, CDCl₃, 20° C.), δ 175.40 (NHCO), 174.10 (NHCO), 157.38 (OCO), 130.86-129.50 (C═C), 58.24, 46.852, 37.69, 32.96, 30.80,30.38, 28.25, 26.80, 23.76, 15.17.

### EXAMPLE 11

### Synthesis of: N,N-dimyristoyl-1,2-diaminopropyl-3-carbamoyl-(aminoethane) 1,2lmp2

Compound 14 (1.75 g, 2.60 mmol) was dissolved in 50 ml of CHCl₃. Ethylenediamine (0.48 ml, 7.78mmol) was added to the reaction mixture while stirring at room temperature for 3 hours. The reaction mixture was taken with 150 mL warm CHCl₃ and transferred to a separation funnel where it was washed with 3×200 ml of warm alkaline brine. The organic layer became clear and the yellow color of 4-nitrophenol disappeared. The organic layer was collected, concentrated and then subjected to column chromatography. The column was eluted successively with 100 ml CHCl₃, 100 ml 1%, 2%, 3%, 5%, 7%, 10%, 15%, 20%, 30%, 60% CH₃OH/CHCl₃. Fractions were collected and tested for the presence of the 1,2lmp2 with TLC and NMR. Fractions containing the product were pooled and evaporated under diminished pressure. Precipitation from CHCl₃: EtOAc (1:5) afforded 0.76 g of 1,2lmp2 (Yield=48%). ¹H NMR (400 MHz, CDCl₃, 20° C., TMS), δ_{H} (ppm): 6.74-6.72 [d, 1H, —CO—NH—CH—], 6.52-6.48 [t, 1H, —CO—NH—CH₂—], 5.39-5.34 [t, 1H, —OCONH—], 4.12-4.09 [m, 3H, —CH—CH₂—OCON—], 3.43-3.30 [m, 2H, —NH—CH₂—CH—], 3.21-3.18 [m, 2H, —OCONH—CH₂—], 2.81-2.78 [m, 2H, —CH₂—NH₂], 2.20-2.10 [m, 4H, —CO—CH₂—], 1.61-1.50 [m, 4H, —CO—CH₂—CH₂—]1.40-1.18 [coherent, 40H, —(CH₂)₁₀—], 0.85-0.82 [m, 6H, —CH₃].

### EXAMPLE 12

### Synthesis of N,N-dimyristoyl-1,2-diaminopropyl-3-carbamoyl-(N,N-Dimethylaminoethane) 1,2lmt2

Compound 14 (1 g, 1.15 mmol) was dissolved in 50 ml of CHCl₃. N,N-Dimethylaminoethane (0.4 g, 4.54 mmol) was added to the reaction mixture while stirring at room temperature for 3 hours. The reaction mixture was taken with addition of 50 ml of CHCl₃ and transferred to a separation funnel where it was washed 3 times with 100 mL of alkaline brine. The organic layer was collected and concentrated then subjected to column chromatography. Fractions were collected and tested for the presence of 1,2lmt2 with TLC and NMR. Fractions containing the product were pooled and evaporated under diminished pressure to give 0.55 g of 1,2lmt2 (Yield=58%). Anal.calcd for C₃₄H₆₈N₄O₄ (MW 588): C, 69.23; H, 11.54; N, 8.97. Found: C, 69.38; H, 11.72; N, 8.94. ¹H NMR (400 MHz, CDCl₃, 20° C., TMS), δ_{H} (ppm): 6.83-6.81 [d, 1H, —CO—NH—CH—], 6.64-6.62 [t, 1H, —CO—NH—CH₂—], 5.61-5.59 [t, 1H, —OCONH—], 4.10-4.08 [m, 3H, —CH—CH₂—OCON—], 3.43-3.32 [m, 2H, —NH—CH₂—CH—], 3.30-3.21 [m, 2H, —OCONH—CH₂—], 2.42-2.38 [m, 2H, —CH₂—N(CH₃)₂], 2.21-2.10 [m, 10H, —CO—CH₂— and N(CH₃)₂], 1.61-1.52 [m, 4H, —CO—CH₂—CH₂—], 1.30-1.18 [coherent, 40H, —(CH₂)₁₀—], 0.85-0.82 [m, 6H, —CH₃].

All other compounds were synthesized using analogous procedures as described above for the dimyristoyl derivatives.

### EXAMPLE 13

### Synthesis of N,N-dilauroyl-1,2-diaminopropyl-3-carbamoyl-(aminoethane) 1,2lmp1

Yield=38.3%. ¹H NMR (400 MHz, CDCl₃, 20° C., TM), δ_{H} (ppm): 6.74-6.72 [d, 1H, —CO—NH—CH—], 6.52-6.48 [t, 1H, —CO—NH—CH₂—], 5.39-5.34 [t, 1H, —OCONH—], 4.12-4.09 [m, 3H, —CH—CH₂—OCON—], 3.43-3.30 [m, 2H, —NH—CH₂—CH—], 3.21-3.18 [m, 2H, —OCONH—CH₂—], 2.81-2.78 [m, 2H, —CH₂—NH₂], 2.20-2.10 [m, 4H, —CO—CH₂—], 1.61-1.50 [m, 4H, —CO—CH₂—CH₂—], 1.40-1.18 [coherent, 48H, —(CH₂)₁₀—], 0.85-0.82 [m, 6H, —CH₃].

### EXAMPLE 14

### Synthesis of N,N(-dilauroyl-1,2-diaminopropyl-3-carbamoyl-(N,N-Dimethylaminoethane) 1,2lmt1

Yield=72.9%. Anal.calcd for C₃₂H₆₄N₄O₄ (MW 560): C, 67.60; H, 11.26; N, 9.86. Found: C, 67.89; H, 11.40; N, 9.65. ¹H NMR (400 MHz, CDCl₃, 20° C., TM), δ_{H} (ppm): 6.74-6.72 [d, 1H, —CO—NH—CH—], 6.52-6.48 [t, 1H, —CO—NH—CH₂—], 5.39-5.34 [t, 1H, —OCONH—], 4.12-4.09 [m, 3H, —CH—CH₂—OCON—], 3.43-3.30 [m, 2H, —NH—CH₂—CH—], 3.21-3.18 [m, 2H, —OCONH—CH₂—], 2.81-2.78 [m, 2H, —CH₂—NH₂], 2.20-2.10 [m, 4H, —CO—CH₂—], 1.61-1.50 [m, 4H, —CO—CH₂—CH₂—], 1.40-1.18 [coherent, 48H, —(CH₂)₁₀—], 0.85-0.82 [m, 6H, —CH₃].

### EXAMPLE 15

### Synthesis of N,N-dipalmitoyl-1,2-diaminopropyl-3-carbamoyl-(aminoethane) 1,2lmp3

Yield=62.5%. ¹H NMR (400 MHz, CDCl₃, 20° C., TM), δ_{H} (ppm): 6.74-6.72 [d, 1H, —CO—NH—CH—], 6.52-6.48 [t, 1H, —CO—NH—CH₂—], 5.39-5.34 [t, 1H, —OCONH—], 4.12-4.09 [m, 3H, —CH—CH₂—OCON—], 3.43-3.30 [m, 2H, —NH—CH₂—CH—], 3.21-3.18 [m, 2H, —OCONH—CH₂—], 2.81-2.78 [m, 2H, —CH₂—NH₂ ], 2.20-2.10 [m, 4H, —CO—CH₂—], 1.61-1.50 [m, 4H, —CO—CH₂—CH₂—), 1.40-1.18 [coherent, 48H, —(CH₂)₁₀—], 0.85-0.82 [m, 6H, —CH₃].

### EXAMPLE 16

### Synthesis of N,N-dipalmitoyl-1,2-diaminopropyl-3-carbamoyl-(N,N-Dimethylaminoethane) 1,2lmt3

Yield=62%. Anal.calcd for C₄₀H₈₀N₄O₄ (MW 672): C, 70.59; H, 11.75; N, 8.24. Found: C, 69.99; H, 11.91; N, 8.10. ¹H NMR (400 MHz, CDCl₃, 20° C., TMS), δ_{H} (ppm): 6.70-6.68 [d, 1H, —CO—NH—CH—], 6.48-6.45 [t, 1H, —CO—NH—CH₂—], 5.37-5.35 [t, 1H, —OCONH—], 4.10-4.08 [m, 3H, —CH—CH₂—OCON—], 3.43-3.32 [m, 2H, —NH—CH₂—CH—], 3.22-3.20 [m, 2H, —OCONH—CH₂—], 2.42-2.38 [m, 2H, —CH₂—N(CH₃)₂], 2.22-2.10 [m, 10H, —CO—CH₂— and N(CH₃)₂], 1.60-1.52 [m, 4H, —CO—CH₂—CH₂—], 1.30-1.18 [coherent, 48H, —(CH₂)₁₀—], 0.85-0.82 [m, 6H, —CH₃].

### EXAMPLE 17

### Synthesis of N,N-disteroyl-1,2-diaminopropyl-3-carbamoyl-(aminoethane) (C₄₂H₈₄N₄O₄) 1,2lmp4

Yield=52.9%. ¹H NMR (400 MHz, CDCl₃, 20° C., TMS), δ_{H} (ppm): 6.74-6.72 [d, 1H, —CO—NH—CH—], 6.52-6.48 [t, 1H, —CO—NH—CH₂—], 5.39-5.34 [t, 1H, —OCONH—], 4.12-4.09 [m, 3H, —CH—CH₂—OCON—], 3.43-3.30 [m, 2H, —NH—CH₂—CH—], 3.21-3.18 [m, 2H, —OCONH—CH₂—], 2.81-2.78 [m, 2H, —CH₂—NH₂], 2.20-2.10 [m, 4H, —CO—CH₂—], 1.61-1.50 [m, 4H, —CO—CH₂—CH₂—], 1.40-1.18 [coherent, 56H, —(CH₂)₁₀—], 0.85-0.82 [m, 6H, —CH₃].

### EXAMPLE 18

### Synthesis of N,N-disteroyl-1,2-diaminopropyl-3-carbamoyl-(N,N-Dimethylaminoethane) 1,2lmt4

Yield=32.9%. Anal.calcd for C₄₄H₈₈N₄O₄ (MW 728): C, 71.74; H, 11.96; N, 7.61. Found: C, 71.6; H, 1214; N, 7.27. ¹H NMR (400 MHz, CDCl₃, 20° C., TMS), δ_{H} —CH₂—N(CH₃)₂], 2.22-2.10 [m, 10H, —CO—CH₂— and —N(CH₃)₂], 2.00-1.90 [m, 8H, —CH₂—CH═CH—CH₂—], 1.62-1.50 [m, 4H, —CO—CH₂—CH₂—], 1.35-1.10 [coherent, 40H, —(CH₂)₁₀—], 0.85-0.70 [m, 6H, —CH₃].

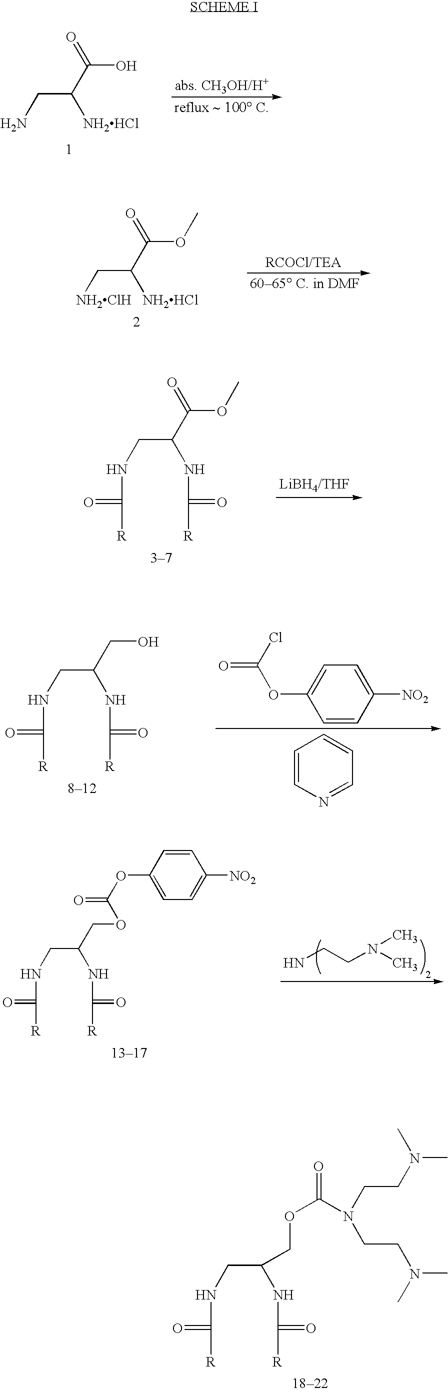

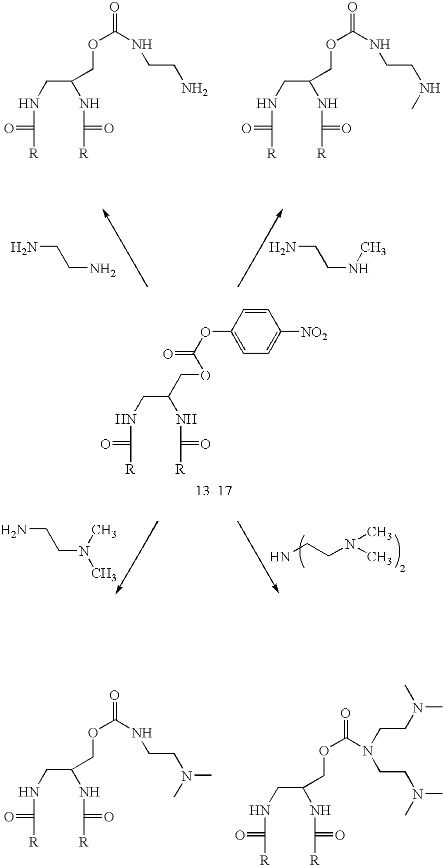

1. Cationic lipids of the general formula S for nucleic acid delivery in vitro and in vivo. 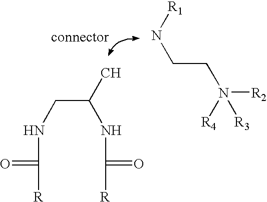
Structure S of cationic lipids for claim 1:
R═C₁₁H₂₃, C₁₃H₂₇, C₁₅H₃₁, C₁₇H₃₅, C₁₇H₃₁
R₁═H, CH₃, CH₂CH₂NH₂, CH₂CH₂NHCH₃, CH₂CH₂N(CH₃)₂, CH₂CH₂NH—C(NH₂)═NH
R₂═H, CH₃
R₃═H, CH₃
R₄═H, CH₃ 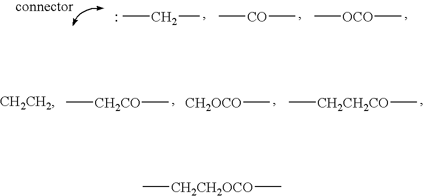
The connector shows how the two parts of structure S can be chemically linked together.

2. The delivery system of claim 1 and any other ingredient that is added to the system of claim 1 for biological activity or any other technological reason.
